(19) United States
(12) Patent Application Publication
(10) Pub. No.: US 2025/0302282 A1
(43) Pub. Date: Oct. 2, 2025

(54) ENDOSCOPE

(71) Applicant: FUJIFILM Corporation, Tokyo (JP)

(72) Inventor: Takashi HARADA, Kanagawa (JP)

(73) Assignee: FUJIFILM Corporation, Tokyo (JP)

(21) Appl. No.: 19/075,716

(22) Filed: Mar. 10, 2025

(30) Foreign Application Priority Data
Mar. 29, 2024 (JP) 2024-055393

(51) Int. Cl. A61B 1/00; A61B 1/018; A61B 90/00

(52) U.S. Cl. CPC A61B 1/00137; A61B 1/00096; A61B 1/00098; A61B 1/00128; A61B 1/00165; A61B 1/018; A61B 90/03; A61B 2090/034

(57) ABSTRACT

An endoscope includes a distal end member that is connected to a distal end side of an insertion part and that is made of a conductive material, and an insulating ring that is mounted on a base end side of the distal end member and that has an insulating property, in which the distal end member has a first region in which an observation window, an illumination window, and a treatment tool outlet port are disposed, and a second region that is provided on a base end side of the first region and on which the insulating ring is mounted, and in a case in which the first region and the second region are projected onto a projection plane perpendicular to a longitudinal axis of the insertion part, a maximum outer diameter of the second region is smaller than a maximum outer diameter of the first region.

### CROSS-REFERENCE TO RELATED APPLICATION

The present application claims priority under 35 U.S.C § 119(a) to Japanese Patent Application No. 2024-055393 filed on Mar. 29, 2024, which is hereby expressly incorporated by reference, in its entirety, into the present application.

### BACKGROUND OF THE INVENTION

### 1. Field of the Invention

The present invention relates to an endoscope, and particularly to an endoscope comprising an annular member having an insulating property at a distal end portion of an insertion part.

### 2. Description of the Related Art

In an endoscope, various treatment tools are introduced from a treatment tool inlet port provided in an operating part, and the treatment tool is led out from a treatment tool outlet port that is open at a distal end portion of an insertion part to the outside and used for a treatment. For example, in the duodenoscope, a treatment tool, such as a contrast tube or a guide wire, is used.

In the duodenoscope, a distal end member constituting the distal end portion of the insertion part is made of a conductive material. Therefore, a distal end cap made of an insulating material, such as a resin, is mounted on the distal end member.

In addition, an annular member made of an insulating material such as a resin is mounted in a region of the distal end member on a base end side with respect to a region on a distal end side in which the distal end cap is mounted. As a result, the distal end portion of the duodenoscope is formed such that the conductive distal end member is covered with the distal end cap having an insulating property and the annular member.

Here, WO2021/152659A discloses an endoscope in which an outer periphery of a distal end member is surrounded by an insulating ring (annular member).

In addition, JP2020-108415A discloses an endoscope in which an annular electrical insulating member is disposed on an outer periphery of a block-shaped distal end constituent portion.

In addition, WO2019/230076A discloses an endoscope in which an insulating ring (annular member) is provided on an outer peripheral portion of a base end portion of a distal end member.

### SUMMARY OF THE INVENTION

In the endoscope, it is desired to reduce (thin out) an outer diameter (diameter) of the distal end portion of the insertion part, but, in the endoscope in which the annular member (insulating ring) is mounted on the distal end member, as in the duodenoscope, the outer diameter of the distal end portion may be determined by the outer diameter of the annular member. That is, in a case in which the outer diameter of the annular member is increased, there is a problem in that the outer diameter of the distal end portion is increased.

The present invention has been made in view of the above-described circumstances, and an object of the present invention is to provide an endoscope in which a diameter of a distal end portion comprising an annular member having an insulating property can be reduced.

A first aspect relates to an endoscope comprising: a distal end member that is connected to a distal end side of an insertion part and that is made of a conductive material; and an annular member that is mounted on a base end side of the distal end member and that has an insulating property, in which the distal end member has a first region in which an observation window, an illumination window, and a treatment tool outlet port are disposed, and a second region that is provided on a base end side of the first region and on which the annular member is mounted, and in a case in which the first region and the second region are projected onto a projection plane perpendicular to a longitudinal axis of the insertion part, a maximum outer diameter of the second region is smaller than a maximum outer diameter of the first region.

A second aspect relates to the endoscope according to the first aspect, in which, in a case in which one of two directions orthogonal to each other on the projection plane is defined as a first direction, and the other of the two directions is defined as a second direction, an outer diameter of the second region in the first direction is smaller than an outer diameter of the first region, and an outer diameter of the second region in the second direction is smaller than an outer diameter of the first region.

A third aspect relates to the endoscope according to the second aspect, in which the second region is included in the first region on the projection plane.

A fourth aspect relates to the endoscope according to any one of the first to third aspects, in which, in a case in which the annular member is projected onto the projection plane, a minimum inner diameter of the annular member is smaller than the maximum outer diameter of the first region.

A fifth aspect relates to the endoscope according to any one of the first to fourth aspects, in which the annular member has deformable rigidity, and the rigidity is rigidity satisfying a condition in which a minimum inner diameter of the annular member is smaller than the maximum outer diameter of the first region even in a case in which the annular member is deformed.

A sixth aspect relates to the endoscope according to any one of the first to fifth aspects, in which the annular member has a shape closed in a circumferential direction as viewed in a direction of the longitudinal axis.

A seventh aspect relates to the endoscope according to any one of the first to sixth aspects, in which the annular member has an annular member inner peripheral surface having a shape corresponding to an outer peripheral surface of the second region, and an annular member outer peripheral surface opposite to the annular member inner peripheral surface, and the annular member outer peripheral surface has an annular protruding portion formed along a circumferential direction, a first outer peripheral surface provided on a base end side with respect to the annular protruding portion, and a second outer peripheral surface provided on a distal end side with respect to the annular protruding portion.

An eighth aspect relates to the endoscope according to the seventh aspect, in which the first outer peripheral surface forms an outer skin mounting surface on which an outer skin of a bendable portion disposed on the base end side of the distal end member is mounted, and the annular protruding portion has an outer skin abutment surface on which a distal end of the outer skin mounted on the outer skin mounting surface abuts.

A ninth aspect relates to the endoscope according to the eighth aspect, further comprising: an outer skin fixing portion that fixes the outer skin mounted on the outer skin mounting surface.

A tenth aspect relates to the endoscope according to the ninth aspect, in which the outer skin fixing portion has a thread winding portion wound around an outer peripheral portion of the outer skin, and an adhesive layer that covers the thread winding portion.

An eleventh aspect relates to the endoscope according to the ninth or tenth aspect, in which an outer diameter of the annular protruding portion is the same as an outer diameter of the outer skin fixing portion.

A twelfth aspect relates to the endoscope according to any one of the seventh to eleventh aspects, further comprising: a distal end cap that is attachably and detachably mounted on the distal end member, in which the second outer peripheral surface has a cap mounting surface on which a base end portion of the distal end cap is mounted, and the annular protruding portion has a cap abutment surface on which a base end of the distal end cap mounted on the cap mounting surface abuts.

A thirteenth aspect relates to the endoscope according to any one of the seventh to twelfth aspects, in which an outer diameter of the annular protruding portion is the same as an outer diameter of the base end portion of the distal end cap.

A fourteenth aspect relates to the endoscope according to any one of the first to thirteenth aspects, in which the distal end member has an annular member abutment surface on which a distal end of the annular member mounted in the second region abuts.

A fifteenth aspect relates to the endoscope according to the fourteenth aspect, in which the distal end member has a flange portion that protrudes in a radial direction between the first region and the second region, and the annular member abutment surface is formed by an end surface on a base end side of the flange portion.

A sixteenth aspect relates to the endoscope according to the fifteenth aspect, further comprising: a distal end cap that is attachably and detachably mounted on the distal end member, in which the distal end cap has a first locking portion that is locked to the flange portion.

A seventeenth aspect relates to the endoscope according to any one of the fourteenth to sixteenth aspects, in which the distal end member has a stepped portion between the first region and the second region, and the annular member abutment surface is formed by an end surface on a base end side of the stepped portion.

An eighteenth aspect relates to the endoscope according to the seventeenth aspect, further comprising: a distal end cap that is attachably and detachably mounted on the distal end member, in which the distal end cap has a second locking portion that is locked to the stepped portion.

A nineteenth aspect relates to the endoscope according to any one of the first to eighteenth aspects, in which the distal end member includes a signal cable that is disposed behind the observation window, a light guide member that is disposed behind the illumination window, and a treatment tool insertion channel through which a treatment tool is inserted.

A twentieth aspect relates to the endoscope according to any one of the first to nineteenth aspects, in which the first region includes an elevator.

According to the aspects of the present invention, it is possible to reduce the diameter of the distal end portion comprising the annular member having the insulating property.

### BRIEF DESCRIPTION OF THE DRAWINGS

FIG. 1 is an explanatory view showing an overall configuration of an endoscope.

FIG. 2 is an enlarged perspective view showing a case in which a distal end portion is viewed from a Z(+) direction side.

FIG. 3 is a perspective view showing a state in which a distal end cap is removed from a distal end member of FIG. 2.

FIG. 4 is a perspective view showing an appearance of the distal end cap.

FIG. 5 is a cross-sectional view showing the distal end cap.

FIG. 6 is a cross-sectional view showing a case in which the distal end portion is cut along a Y-Z plane.

FIG. 7 is a perspective view showing the distal end member as viewed from a Y(+) direction side.

FIG. 8 is a perspective view showing a state in which a cover shown in FIG. 7 is removed from the distal end member.

FIG. 9 is a perspective view showing the distal end member as viewed from the Y(+) direction side.

FIG. 10 is a side view showing the distal end member shown in FIG. 9 as viewed from an X(+) direction side.

FIG. 11 is a cross-sectional view showing main parts of a fixing structure of an angle rubber.

FIG. 12 is an enlarged cross-sectional view showing main parts of the fixing structure shown in FIG. 11.

### DESCRIPTION OF THE PREFERRED EMBODIMENTS

Hereinafter, an endoscope according to an embodiment of the present invention will be described with reference to the accompanying drawings.

### Overall Configuration of Endoscope 1

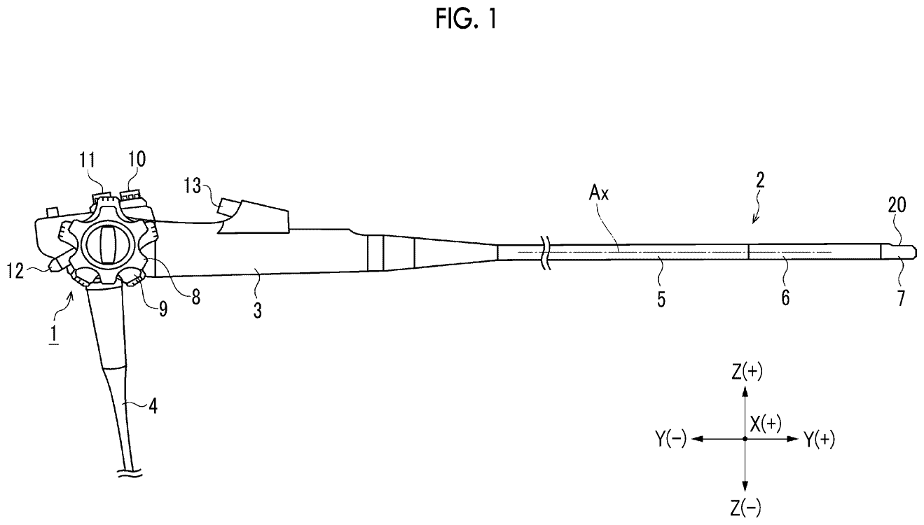

FIG. 1 is an overall configuration view showing an endoscope 1 according to the present embodiment.

As shown in FIG. 1, the endoscope 1 comprises an insertion part 2 that is inserted into a subject, and an operating part 3 that is connected to a base end portion of the insertion part 2 and is operated by an operator. It should be noted that, in the embodiment, a side-viewing endoscope used as a duodenoscope is described as an example.

Hereinafter, in a case of describing a configuration of each part of the endoscope 1, a three-dimensional orthogonal coordinate system of X, Y, and Z will be used for convenience of description. A Z direction in the drawings is an up-down direction, a Z(+) direction side indicates an up direction, and a Z(−) direction side indicates a down direction. In addition, an X direction in the drawings is a direction perpendicular to the Z direction, an X(+) direction side indicates a right direction, and an X(−) direction side indicates a left direction. In addition, a Y direction in the drawings is a direction perpendicular to both the Z direction and the X direction, a Y(+) direction side indicates a distal end side direction, and a Y(−) direction side indicates a base end side direction. It should be noted that, in addition, each of the above-described directions refers to a direction in a case in which the operator views the insertion part 2 from an upper side in a case in which the operator grips the operating part 3 of the endoscope 1.

### Insertion Part 2

The insertion part 2 of the endoscope 1 is inserted into the subject through an oral cavity and is further inserted into a duodenum from an esophagus via a stomach. Accordingly, a procedure such as an examination or a treatment of the duodenum is performed by using a treatment tool inserted into the insertion part 2.

Examples of the treatment tool include biopsy forceps having a cup that can collect a biological tissue at a distal end portion thereof, an endoscopic sphincterotomy (EST) knife, a contrast tube, and a guide wire.

The insertion part 2 has a longitudinal axis Ax, and has a soft portion 5, a bendable portion 6, and a distal end portion 7 in order from a base end side to a distal end side. It should be noted that a longitudinal axis Ax direction and the Y direction are the same direction.

The soft portion 5 occupies most of the insertion part 2 from the base end side, and is formed by, for example, a metal spiral tube and a net (not shown), and is further formed by a resin tube covering the outer periphery of the metal spiral tube and the net. The soft portion 5 has flexibility that allows the soft portion 5 to be bent in any direction, and is bent along an insertion path into a body cavity in a case in which the insertion part 2 is inserted into the body cavity.

The bendable portion 6 is disposed on the distal end side of the soft portion 5 and on the base end side of a distal end member 20 of the distal end portion 7. The bendable portion 6 has a tubular body in which a plurality of angle rings are connected to each other in a swingable manner, and is formed by covering an outer periphery of the tubular body with a mesh body woven with a metal wire and further covering the outer periphery of the mesh body with angle rubber made of rubber, which is an outer skin. In addition, a plurality of wires extend from the angle knobs 8 and 9 of the operating part 3 to the bendable portion 6, and distal end portions of these wires are fixed to the distal end portion of the tubular body. Accordingly, the bendable portion 6 is bent in the up-down direction (Z direction) and the left-right direction (X direction) in response to a rotational movement operation of the angle knobs 8 and 9.

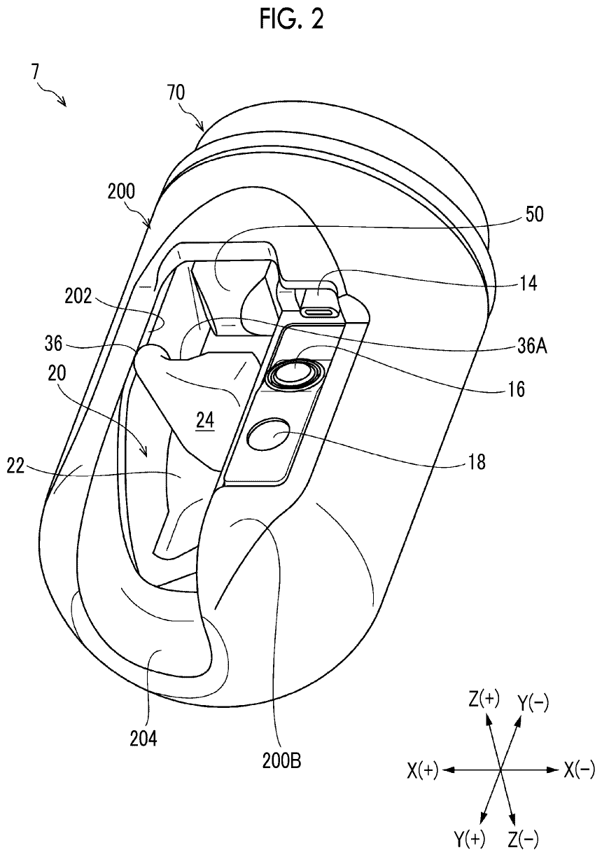

Although the distal end portion 7 will be described in detail below with reference to FIG. 2 and the following drawings, the distal end portion 7 has the distal end member 20 and a distal end cap 200 (see FIG. 2) that is attachably and detachably mounted on the distal end member 20.

The distal end member 20 is made of a conductive material having corrosion resistance. The distal end cap 200 is made of an insulating and elastic material, for example, silicon rubber. The distal end member 20 is a member connected to the distal end side of the insertion part 2, and is an example of a distal end member according to the embodiment of the present invention. Further, the distal end cap 200 is an example of a distal end cap according to the embodiment of the present invention.

### Operating Part 3

As shown in FIG. 1, the operating part 3 includes angle knobs 8 and 9 for bending the bendable portion 6, an air/water supply button 10, a suction button 11, an elevating operation lever 12, and a treatment tool inlet port 13.

The air/water supply button 10 is an operation button for performing air/water supply from an air/water supply nozzle 14 (see FIG. 2) of the distal end portion 7 toward an observation window 16, an illumination window 18, and the like via an air/water supply channel (not shown) inserted into the insertion part 2.

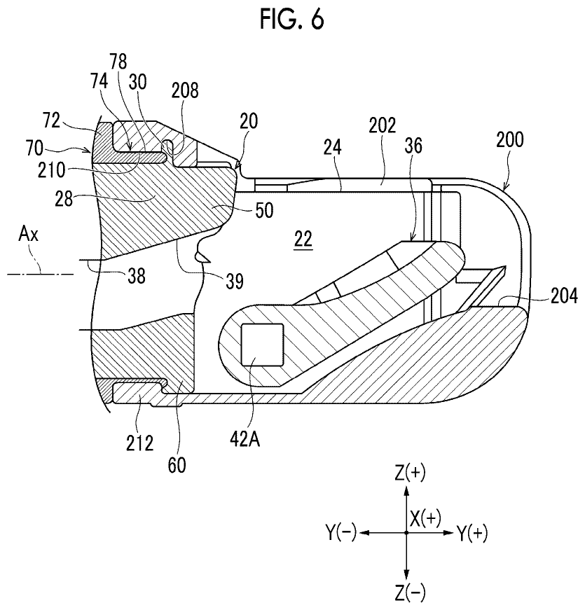

The suction button 11 is an operation button for suctioning a body fluid and the like via a treatment tool insertion channel 38 (see FIG. 6) inserted into the insertion part 2.

The elevating operation lever 12 is a lever for performing an operation of elevating and lowering an elevator 36 (see FIG. 2) provided at the distal end portion 7. The elevating operation lever 12 is one of constituent members of a treatment tool elevating mechanism.

The treatment tool inlet port 13 communicates with the treatment tool insertion channel 38, and is an opening through which the operator introduces the treatment tool or the like. The treatment tool introduced from the treatment tool inlet port 13 is guided to the distal end portion 7 through the treatment tool insertion channel 38.

A universal cord 4 is connected to the operating part 3, and the universal cord 4 is connected to an image processing device and a light source device (not shown). A monitor device (not shown) is connected to the image processing device via a cable. The overall structure of the endoscope 1 is as described above. Hereinafter, a structure of the distal end portion 7 will be described.

### Structure of Distal End Portion 7

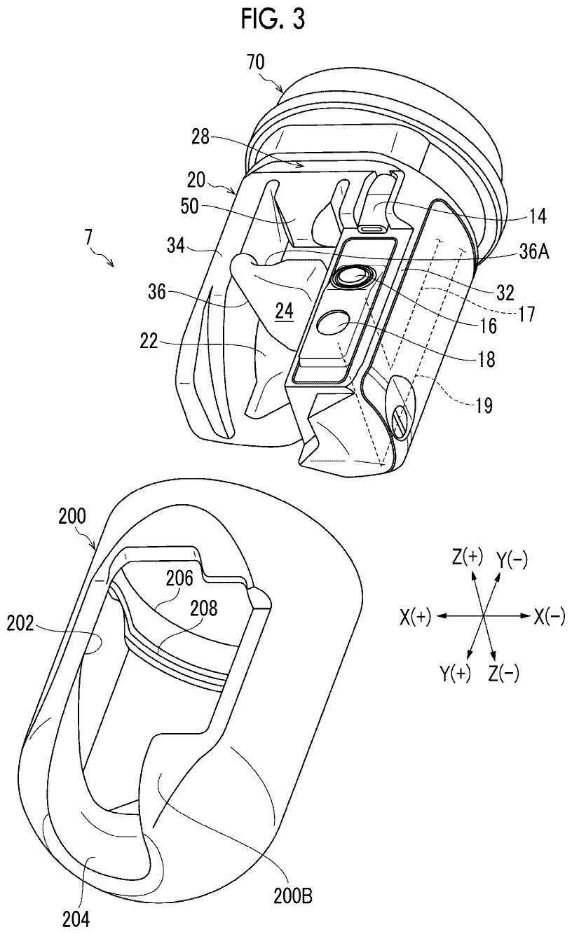

FIG. 2 is an enlarged perspective view showing a case in which the distal end portion 7 is viewed from the Z (+) direction side. FIG. 2 shows a state in which the distal end cap 200 is mounted on the distal end member 20. In addition, FIG. 3 is a perspective view showing a state in which the distal end cap 200 is removed from the distal end member 20 of FIG. 2.

As shown in FIG. 2, in a state in which the distal end cap 200 is mounted on the distal end member 20, the distal end cap 200 has an upper opening 202 that communicates with an opening 24 on the Z(+) direction side of an elevator housing portion 22 to be described later. Subsequently, a configuration of the distal end cap 200 will be described.

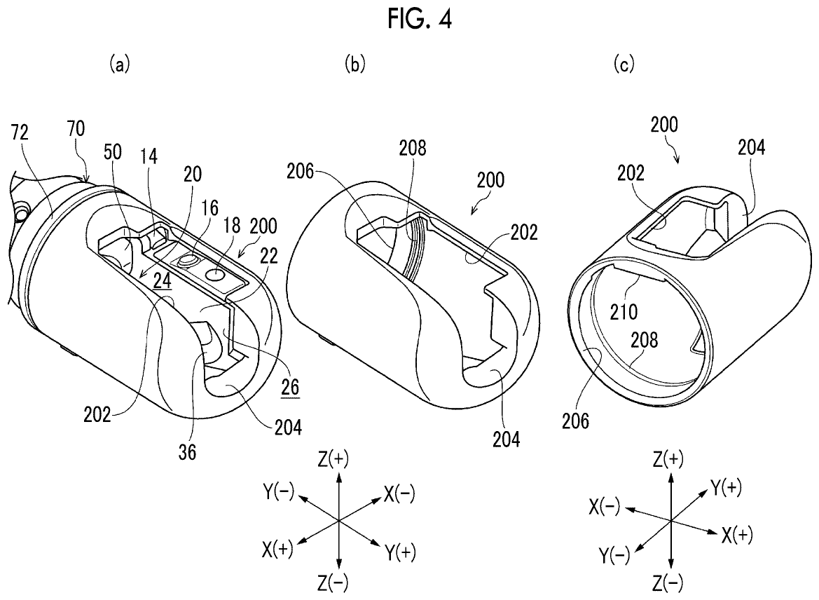

FIG. 4 is a perspective view showing an appearance of the distal end cap 200. In FIG. 4, (a) is a perspective view showing a state in which the distal end cap 200 is mounted on the distal end member 20 as viewed from the Y(+) direction side, (b) is a perspective view showing the distal end cap 200 as viewed from the Y(+) direction side, and (c) is a perspective view showing the distal end cap 200 as viewed from the Y(−) direction side.

As shown in FIG. 4, the distal end cap 200 is formed in a substantially tubular shape to house the distal end member 20. In addition, the distal end cap 200 has an upper opening 202 that communicates with the opening 24 of the elevator housing portion 22, a distal end portion opening 204 that communicates with a distal end opening 26 of the elevator housing portion 22 on the Y(+) direction side, and a base end portion opening 206 for mounting the distal end cap 200 on the distal end member 20.

The upper opening 202 is provided on the Z(+) direction side, and is formed to have a size at which the distal end portion of the air/water supply nozzle 14, the observation window 16, the illumination window 18, and a guide wire fixing portion 50 described later are exposed in addition to the elevator housing portion 22 (opening 24). In addition, the distal end portion opening 204 is installed consecutively to the upper opening 202, and is formed as an opening for air venting for preventing a body cavity wall from being suctioned to the upper opening 202 during a suction operation of the endoscope 1. In addition, the base end portion opening 206 is an opening for inserting the distal end member 20.

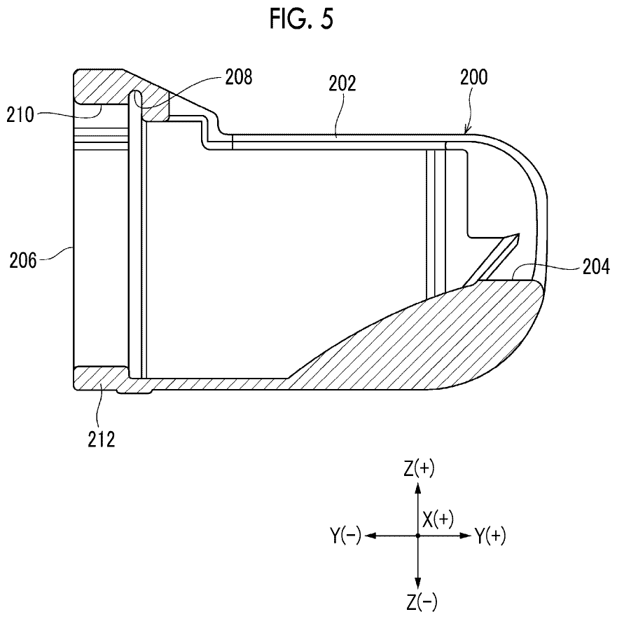

FIG. 5 is a cross-sectional view showing the distal end cap 200 in a case in which the distal end cap 200 is cut along a Y-Z plane. FIG. 6 is a cross-sectional view showing a case in which the distal end portion 7 shown in FIG. 2 is cut along the Y-Z plane.

As shown in FIGS. 5 and 6, the distal end cap 200 has an engaging groove 208, a flat portion 210, and a hook portion 212 on an inner peripheral surface on the base end side. The engaging groove 208 is provided over the entire inner peripheral surface of the distal end cap 200. The flat portion 210 is a surface of the inner peripheral surface of the distal end cap 200 on the Z(+) direction side, and is provided on a surface on the base end side (Y(−) direction side) with respect to the engaging groove 208. The surface of the flat portion 210 is, for example, substantially parallel to an X-Y plane. The hook portion 212 is formed as a protruding portion that is a surface of the inner peripheral surface of the distal end cap 200 on the Z(−) side and protrudes in the Z(+) direction.

As shown in FIG. 6, the distal end member 20 has a flange portion 30 and a stepped portion 60. The flange portion 30 is provided to protrude in a radial direction from an upper surface (surface on the Z(+) direction side) of the distal end member 20. The stepped portion 60 is provided to protrude in the radial direction from a lower surface (surface on the Z(−) direction side) of the distal end member 20.

As shown in FIG. 6, in a case in which the distal end cap 200 is mounted on the distal end member 20, the engaging groove 208 of the distal end cap 200 is locked to the flange portion 30 of the distal end member 20, and the flat portion 210 abuts on the flat portion 78 provided on an insulating ring 70 described later. The hook portion 212 is locked to the stepped portion 60. Therefore, the distal end cap 200 is reliably mounted on the distal end member 20. It should be noted that the flange portion 30 and the engaging groove 208 are examples of a flange portion and a first locking portion according to the embodiment of the present invention, respectively. In addition, the stepped portion 60 and the hook portion 212 are examples of a stepped portion and a second locking portion according to the embodiment of the present invention, respectively. Subsequently, a configuration of the distal end member 20 will be described.

As shown in FIG. 3, the distal end member 20 has a first wall portion 32 and a second wall portion 34 that face each other in the X direction. The first wall portion 32 and the second wall portion 34 are installed consecutively to a base portion 28 of the distal end member 20 at the respective base ends thereof, and extend from the base portion 28 to the Y(+) direction side.

Further, the elevator housing portion 22 for housing the elevator 36 is provided between the first wall portion 32 and the second wall portion 34 in the X direction. The elevator housing portion 22 is formed as a slit-shaped space along the Y direction.

A treatment tool outlet port 39 (see FIG. 6) is provided in the distal end member 20. The distal end side of the treatment tool insertion channel 38 (see FIG. 6) communicates with the treatment tool outlet port 39. The treatment tool insertion channel 38 is inserted into the insertion part 2 (see FIG. 1) from the distal end member 20, and the base end side of the treatment tool insertion channel 38 is connected to the treatment tool inlet port 13 of the operating part 3.

Therefore, the treatment tool introduced from the treatment tool inlet port 13 is guided to the elevator housing portion 22 through the treatment tool insertion channel 38 and the treatment tool outlet port 39. The treatment tool guided to the elevator housing portion 22 is changed in a lead-out direction by the elevator 36. It should be noted that the treatment tool insertion channel 38 and the treatment tool outlet port 39 are examples of a treatment tool insertion channel and a treatment tool outlet port according to the embodiment of the present invention.

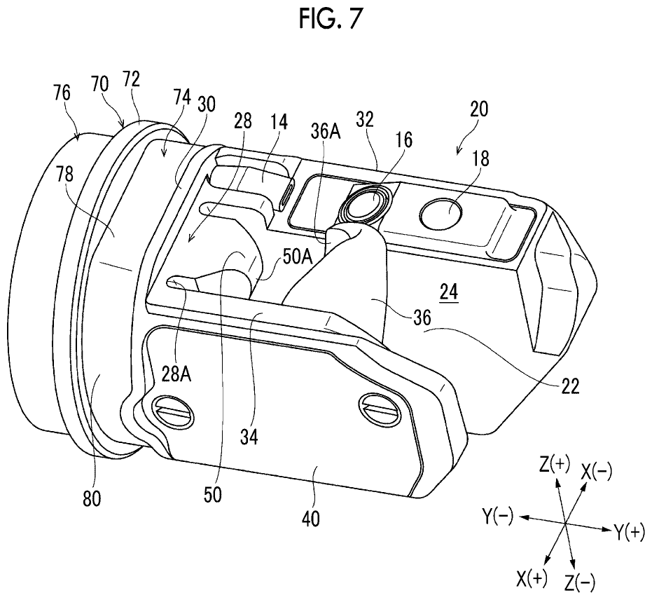

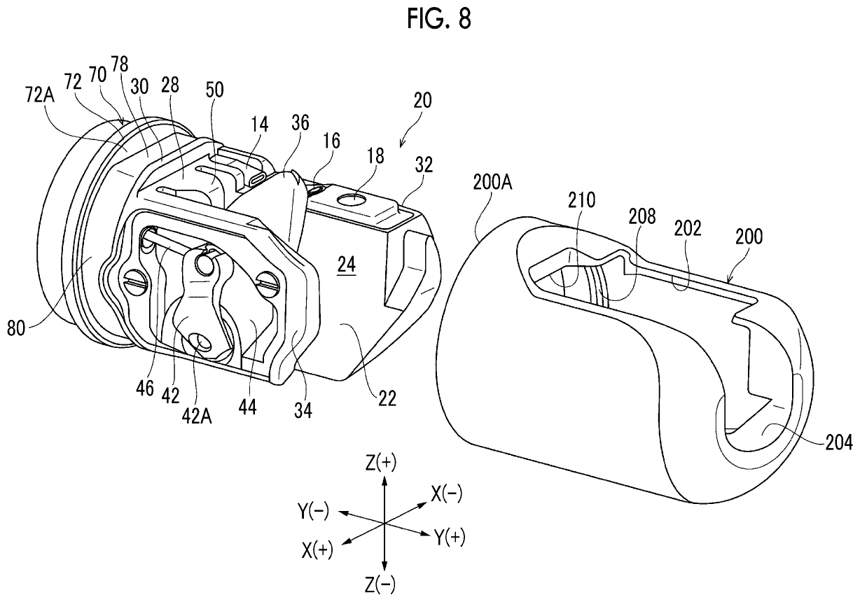

FIG. 7 is a perspective view showing the distal end member 20 as viewed from the Y(+) direction side. FIG. 8 is a perspective view showing a state in which a cover 40 shown in FIG. 7 is removed from the second wall portion 34, and in FIG. 8, the distal end cap 200 is also shown. First Wall Portion 32

As shown in FIGS. 7 and 8, the observation window 16 and the illumination window 18 are provided on the first wall portion 32. An imaging unit and an illumination unit are provided in the first wall portion 32.

The imaging unit has an imaging optical system disposed behind (Z(−) direction side of) the observation window 16 and a complementary-metal-oxide semiconductor (CMOS) type or charge-coupled device (CCD) type imaging element. The imaging element is connected to the image processing device via a signal cable 17 (see FIG. 3) inserted into the insertion part 2 from the first wall portion 32. An imaging signal of a subject image obtained by the imaging unit is input to the image processing device through the signal cable 17, and is displayed as the subject image on the monitor connected to the image processing device through the cable. It should be noted that the observation window 16 and the signal cable 17 are examples of an observation window and a signal cable according to the embodiment of the present invention.

The illumination unit has an illumination lens installed behind (Z(−) direction side of) the illumination window 18 and a light guide 19 (see FIG. 3) disposed such that a distal end thereof faces the illumination lens. The light guide 19 is inserted into the insertion part 2 from the first wall portion 32, and a base end portion of the light guide 19 is connected to the light source device. As a result, irradiation light from the light source device is transmitted through the light guide 19 and is emitted from the illumination window 18. It should be noted that the illumination window 18 and the light guide 19 are examples of an illumination window and a light guide member according to the embodiment of the present invention.

### Second Wall Portion 34

As shown in FIG. 8, a lever housing chamber 44 that has a recessed shape and that houses a lever 42 is provided in the second wall portion 34. Among two wall surfaces of the second wall portion 34 facing each other in the X direction, the lever housing chamber 44 is provided on a wall surface on a side opposite to a wall surface that faces the elevator housing portion 22.

The lever 42 in the lever housing chamber 44 is provided with a rotation shaft 42A at one end portion thereof. The rotation shaft 42A penetrates the second wall portion 34 in the X direction, and is connected to the elevator 36. Further, the other end portion of the lever 42 is connected to an operating wire 46. The operating wire 46 is inserted into the insertion part 2 (see FIG. 1), and is connected to the elevating operation lever 12 of the operating part 3.

Therefore, the operator operates the elevating operation lever 12 to push or pull the operating wire 46, and the lever 42 swings in conjunction with the push-pull operation. Accordingly, the elevator 36 swings using the rotation shaft 42A as a swing shaft, and a position of the elevator 36 is changed between an elevated position and a fallen position. Here, the treatment tool elevating mechanism is formed by the lever 42 having the rotation shaft 42A, the operating wire 46, and the elevating operation lever 12. It should be noted that the lever housing chamber 44 is sealed by the cover 40 shown in FIG. 7.

### Elevator 36

As shown in FIGS. 7 and 8, the elevator 36 is provided in the distal end member 20, and is housed in the elevator housing portion 22. In addition, the elevator 36 is attached to the second wall portion 34 swingably via the rotation shaft 42A. The elevator 36 is driven by the treatment tool elevating mechanism to perform an elevating operation and a falling operation. It should be noted that the elevator 36 is an example of an elevator according to the embodiment of the present invention.

The elevator 36 has a guide surface 36A (see FIG. 2 and the like). The guide surface 36A is formed on a surface that faces the Z(+) direction side in a case in which the elevator 36 is located at the fallen position. The guide surface 36A is a surface for guiding the treatment tool, and is formed in a substantially U-shape that is recessed inward from the base end side that is a swing end of the elevator 36 to the distal end side.

In a case in which the elevator 36 is elevated by the treatment tool elevating mechanism, the treatment tool guided to the elevator housing portion 22 is guided by the guide surface 36A of the elevator 36 in a direction toward the opening 24 of the elevator housing portion 22 and is led out from the upper opening 202 of the distal end cap 200 to the outside. In addition, the lead-out direction of the treatment tool can be changed by adjusting an elevating angle of the elevator 36 by the treatment tool elevating mechanism.

### Guide Wire Fixing Portion 50

As shown in FIG. 7, a guide wire fixing portion 50 is provided at the base portion 28. The guide wire fixing portion 50 protrudes from a distal end wall portion 28A of the base portion 28 toward the elevator housing portion 22 on the Y(+) direction side.

The guide wire fixing portion 50 is formed as a tapered protruding portion in the Y(+) direction. In other words, the guide wire fixing portion 50 is formed to have a substantially trapezoidal cubic shape with the distal end wall portion 28A as a bottom surface.

In a case in which the elevator 36 is elevated to the maximum elevated position, a guide wire (not shown) guided from the treatment tool insertion channel 38 to the elevator housing portion 22 is interposed and fixed between the protruding distal end portion 50A of the guide wire fixing portion 50 and the guide surface 36A having a recessed shape of the elevator 36. It should be noted that the guide wire fixing portion 50 is installed adjacent to the air/water supply nozzle 14 in the X direction.

### First Region 100 and Second Region 110

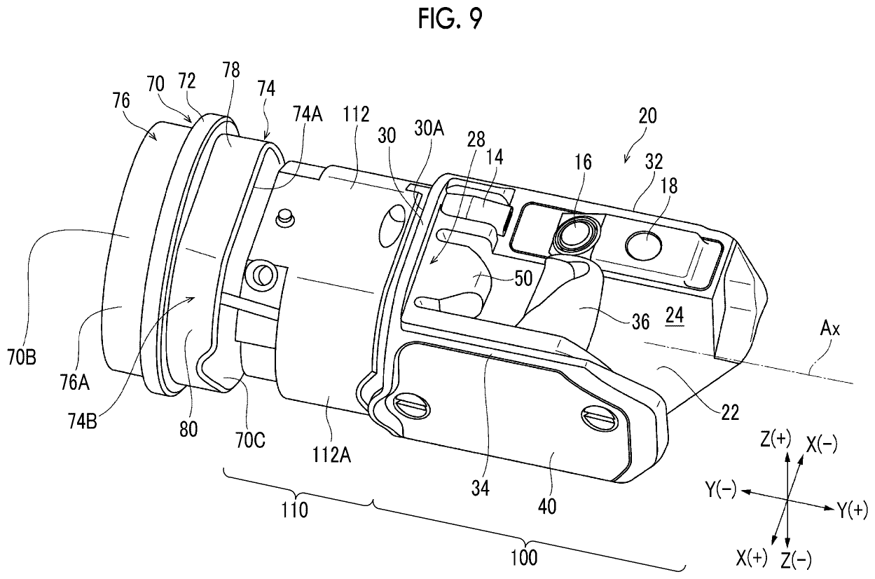

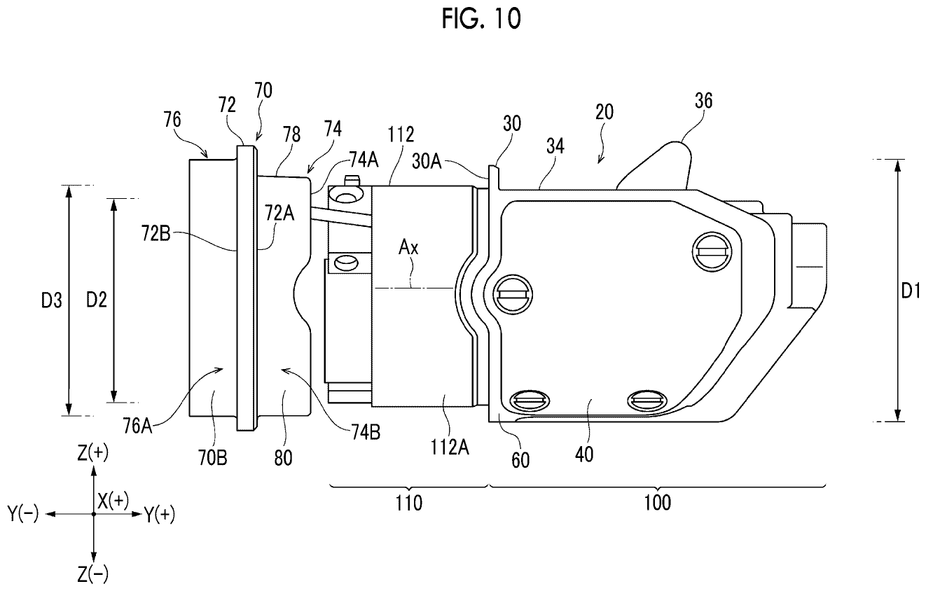

FIG. 9 is a perspective view showing the distal end member 20 as viewed from the Y(+) direction side. FIG. 10 is a side view showing the distal end member 20 shown in FIG. 9 as viewed from the X(+) direction side. It should be noted that FIGS. 9 and 10 show a state in which the insulating ring 70 is separated from the second region 110 to the Y(−) direction side.

As shown in FIGS. 9 and 10, the distal end member 20 formed as described above has a first region 100 and a second region 110. The first region 100 and the second region 110 are examples of a first region and a second region according to the embodiment of the present invention, respectively.

The first region 100 is a region of the distal end side portion of the distal end member 20 in the longitudinal axis Ax direction. The second region 110 is a region on the base end side with respect to the first region 100, which is a region of the base end side portion of the distal end member 20. The flange portion 30 and the stepped portion 60 are provided between the first region 100 and the second region 110. That is, in the distal end member 20 of the present example, with the flange portion 30 and the stepped portion 60 as a boundary, the first region 100 is provided on the distal end side, and the second region is provided on the base end side.

The observation window 16, the illumination window 18, the treatment tool outlet port 39, and the elevator 36 are included in the first region 100. As shown in FIG. 10, the first region 100 has a maximum outer diameter (diameter) D1. The maximum outer diameter D1 refers to the maximum outer diameter in a case in which the first region 100 is projected onto a projection plane (X-Z plane) perpendicular to the longitudinal axis Ax.

Meanwhile, the second region 110 is a region in which the insulating ring 70 is mounted, and has a maximum outer diameter (diameter) D2 smaller than the maximum outer diameter D1 of the first region 100. The maximum outer diameter D2 refers to the maximum outer diameter in a case in which the second region 110 is projected onto the projection plane (X-Z plane) perpendicular to the longitudinal axis Ax.

That is, as shown in FIG. 10, in a case in which the first region 100 and the second region 110 are projected on the projection plane (X-Z plane), the maximum outer diameter D2 of the second region 110 is formed to be smaller than the maximum outer diameter D1 of the first region 100.

In addition, in the projection plane (X-Z plane), among two directions (X direction and Z direction) orthogonal to each other, the outer diameter of the second region 110 in the X direction is smaller than the outer diameter of the first region 100, and the outer diameter of the second region 110 in the Z direction is smaller than the outer diameter of the first region 100. It should be noted that the X direction and the Z direction are examples of a first direction and a second direction according to the embodiment of the present invention.

Further, the second region 110 is included in the first region 100 in the projection plane (X-Z plane).

The insulating ring 70 is mounted in the second region 110 formed as described above. The insulating ring 70 is formed in a ring shape that is closed in a circumferential direction as viewed in the longitudinal axis Ax direction. It should be noted that the insulating ring 70 is an example of an annular member according to the embodiment of the present invention. Hereinafter, the insulating ring 70 will be described.

### Insulating Ring 70

As shown in FIGS. 9 and 10, the second region 110 has a cylindrical portion 112 along the longitudinal axis Ax, and the insulating ring 70 is mounted on an outer peripheral portion 112A thereof. The insulating ring 70 is made of an insulating material, such as plastic or ceramic.

As shown in FIG. 10, in a case in which the insulating ring 70 is projected onto the projection plane (X-Z plane) perpendicular to the longitudinal axis Ax, a minimum inner diameter D3 of the insulating ring 70 is formed to be smaller than the maximum outer diameter D1 of the first region 100. The minimum inner diameter D3 is naturally formed to be larger than the maximum outer diameter D2 of the second region 110.

In addition, in a case in which the insulating ring 70 is formed by a deformable rigid member such as plastic, the rigidity satisfies a condition in which the minimum inner diameter D3 is smaller than the maximum outer diameter D1 even in a case in which the insulating ring 70 is deformed. Therefore, the insulating ring 70 cannot be inserted into the first region 100, but can be inserted into the second region 110.

The insulating ring 70 has an insulating ring inner peripheral surface 70C and an insulating ring outer peripheral surface 70B opposite to the insulating ring inner peripheral surface 70C. The insulating ring inner peripheral surface 70C has a shape (substantially the same shape or a similar shape) corresponding to the outer peripheral portion 112A of the cylindrical portion 112 of the second region 110. It should be noted that the insulating ring inner peripheral surface 70C and the insulating ring outer peripheral surface 70B are examples of an inner peripheral surface of the insulating member and an outer peripheral surface of the insulating member according to the embodiment of the present invention, respectively.

The insulating ring outer peripheral surface 70B has a flange portion 72 formed along the circumferential direction, a cap mounting portion 74 provided on the distal end side with respect to the flange portion 72, and a bendable portion mounting portion 76 disposed on the base end side with respect to the flange portion 72. It should be noted that the flange portion 72 is an example of an annular protruding portion according to the embodiment of the present invention.

### Cap Mounting Portion 74

The cap mounting portion 74 has an outer peripheral surface 74B. The outer peripheral surface 74B has a flat portion 78 and an arc portion 80 and is provided on the distal end side with respect to the flange portion 72. It should be noted that the outer peripheral surface 74B is an example of a second outer peripheral surface according to the embodiment of the present invention.

The flat portion 78 is provided on a surface of the outer peripheral surface 74B on the Z(+) side. The flat portion 78 is disposed at a position (position on the Z(−) side) lower than an upper end of the flange portion 30 of the distal end member 20 in a case in which the insulating ring 70 is mounted in the second region 110. A surface of the flat portion 78 is, for example, substantially parallel to the X-Y plane. It should be noted that the arc portion 80 is formed at a position on the outer peripheral surface 74B excluding the flat portion 78.

The outer peripheral surface 74B forms a cap mounting surface on which the base end portion of the distal end cap 200 is mounted. That is, the outer peripheral surface 74B of the insulating ring 70 has the cap mounting surface.

In a case in which the insulating ring 70 is mounted in the second region 110, first, the cap mounting portion 74 of the insulating ring 70 is externally mounted from the base end side to the distal end side of the second region 110 (cylindrical portion 112). Then, a distal end 74A of the insulating ring 70 (cap mounting portion 74) abuts on an end surface 30A of the flange portion 30 on the base end side.

As a result, the insulating ring 70 is mounted in the second region 110. It should be noted that the end surface 30A of the flange portion 30 on the base end side, on which the distal end 74A of the insulating ring 70 abuts, is an example of an annular member abutment surface according to the embodiment of the present invention.

In addition, in a case in which the insulating ring 70 is mounted in the second region 110, the outer peripheral surface 74B (flat portion 78 and arc portion 80) that is the cap mounting surface is disposed on the base end side of the flange portion 30 (see FIGS. 7 and 8).

Subsequently, in a case in which the distal end cap 200 is mounted on the distal end member 20, the distal end cap 200 is externally mounted from the distal end side to the base end side of the first region 100 (first wall portion 32 and second wall portion 34), and a base end 200A (see FIG. 8) of the distal end cap 200 abuts on the end surface 72A of the flange portion 72 of the insulating ring 70 on the distal end side.

As a result, as shown in FIG. 6, the base end portion of the distal end cap 200 is mounted on the outer peripheral surface 74B of the insulating ring 70. It should be noted that the end surface 72A of the flange portion 72 on the distal end side, which abuts on the base end 200A of the distal end cap 200, is an example of a cap abutment surface according to the embodiment of the present invention.

In addition, as shown in FIG. 6, in a case in which the base end portion of the distal end cap 200 is mounted on the outer peripheral surface 74B of the insulating ring 70, the engaging groove 208 of the distal end cap 200 is locked to the flange portion 30 of the distal end member 20, and the flat portion 210 of the distal end cap 200 abuts on the flat portion 78 of the insulating ring 70. Further, the hook portion 212 of the distal end cap 200 is locked to the stepped portion 60 of the distal end member 20. As a result, the distal end member 20 made of a metal is covered with the distal end cap 200 having an insulating property and the insulating ring 70.

With such a configuration, with the endoscope 1 according to the present embodiment, the insulating property of the distal end portion 7 can be ensured by the distal end cap 200 and the insulating ring 70. Further, the locking of the flange portion 30 and the engaging groove 208 and the locking of the stepped portion 60 and the hook portion 212 can prevent the distal end cap 200 from coming off the distal end member 20 to the Y(+) direction side. In addition, since the flat portion 78 and the flat portion 210 abut on each other, it is possible to prevent the distal end cap 200 from being displaced with respect to the distal end member 20 in a direction around the longitudinal axis Ax.

### Bendable Portion Mounting Portion 76

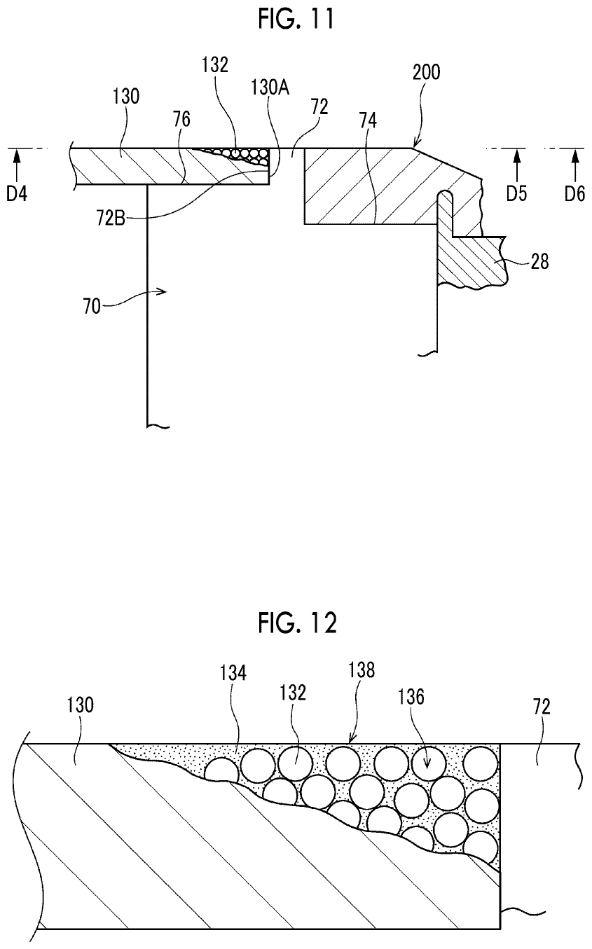

Meanwhile, the bendable portion mounting portion 76 of the insulating ring 70 has an outer peripheral surface 76A as shown in FIGS. 9 and 10. The outer peripheral surface 76A is provided on the base end side with respect to the flange portion 72 and forms an outer skin mounting surface on which an angle rubber 130 (see FIG. 11) of the bendable portion 6 is mounted. It should be noted that the outer peripheral surface 76A is an example of a first outer peripheral surface according to the embodiment of the present invention, and the angle rubber 130 is an example of an outer skin according to the embodiment of the present invention.

FIG. 11 is a cross-sectional view showing main parts of a fixing structure of the angle rubber 130 to the bendable portion mounting portion 76. FIG. 12 is an enlarged cross-sectional view showing main parts of the fixing structure shown in FIG. 11.

As shown in FIG. 11, a distal end portion of the angle rubber 130 is mounted on the bendable portion mounting portion 76, and an end surface of the angle rubber 130 on the distal end 130A side is brought close to an end surface 72B on the base end side of the flange portion 72. As a result, the angle rubber 130 can be mounted on the bendable portion mounting portion 76. It should be noted that, in a case in which the angle rubber 130 is mounted on the bendable portion mounting portion 76, the end surface of the angle rubber 130 on the distal end 130A side may abut on the end surface 72B of the flange portion 72 on the base end side.

Thereafter, the angle rubber 130 is fixed to the insulating ring 70 by winding a thread 132 around the distal end portion thereof. As shown in FIG. 12, the thread 132 is fixed to the angle rubber 130 with an adhesive 134. It should be noted that the distal end portion of the angle rubber 130 has a thread winding portion 136 around which the thread 132 is wound and an adhesive layer 138 that covers the thread winding portion 136. The thread winding portion 136 and the adhesive layer 138 are examples of an outer skin fixing portion according to the embodiment of the present invention, and are examples of a thread winding portion and an adhesive layer according to the embodiment of the present invention.

In addition, as shown in FIG. 11, an outer diameter (diameter) D4 of the adhesive layer 138 and an outer diameter D5 of the flange portion 72 are the same as each other. As a result, the insulating ring 70 and the angle rubber 130 are fixed to each other without a step difference therebetween on the respective outer peripheral surfaces.

In addition, as shown in FIG. 11, an outer diameter (diameter) D6 of the base end portion of the distal end cap 200 and the outer diameter D5 of the flange portion 72 are the same as each other. As a result, the distal end cap 200 and the insulating ring 70 are fixed to each other without a step difference therebetween on the respective outer peripheral surfaces.

Therefore, the distal end cap 200, the insulating ring 70, and the angle rubber 130 are fixed to each other without a step difference therebetween on the respective outer peripheral surfaces.

It should be noted that the same outer diameter does not always mean that the outer diameters are completely the same as each other. A case in which there is a slight variation in the outer diameters due to assembly errors during manufacturing or individual differences of the respective members and a case of a step difference that is considered to be almost the same as 1 mm or less are also included in a range in which the outer diameters are the same as each other.

In the endoscope, there is a problem in that the outer diameter of the distal end portion increases due to the outer diameter of the insulating ring as described above.

Therefore, in order to reduce the outer diameter of the distal end portion 7, the endoscope 1 according to the present embodiment adopts the configuration in which the distal end member 20 of the distal end portion 7 has the first region 100 having the maximum outer diameter D1 and the second region 110 that is provided on the base end side with respect to the first region 100 and that has the maximum outer diameter D2 smaller than the maximum outer diameter D1, and the insulating ring 70 is mounted in the second region 110.

Here, one comparative example will be described. For example, in a case in which the maximum outer diameter D2 of the second region 110 is larger than the maximum outer diameter D1 of the first region 100, the insulating ring mounted in the second region 110 should have a minimum inner diameter larger than the maximum outer diameter D2 of the second region 110. In this case, from the viewpoint of ensuring the strength of the insulating ring, the outer diameter of the insulating ring should be increased as the minimum inner diameter of the insulating ring is increased, so that the outer diameter of the insulating ring is increased. As a result, the outer diameter (diameter) of the distal end portion determined by the outer diameter of the insulating ring is also increased, which causes the increase in the diameter of the distal end portion.

On the other hand, in the endoscope 1 according to the present embodiment, since the maximum outer diameter D2 of the second region 110 is smaller than the maximum outer diameter D1 of the first region 100, it is not necessary to increase the minimum inner diameter and the outer diameter of the insulating ring 70 as compared with the above-described comparative example. As a result, it is possible to reduce the outer diameter of the distal end portion 7.

Therefore, the endoscope 1 according to the present embodiment can reduce the diameter of the distal end portion 7 comprising the insulating ring 70.

In addition, since the endoscope 1 according to the present embodiment has a configuration (insertion configuration) in which the insulating ring 70 is mounted in the second region 110 from the base end side of the second region 110, for example, there is an advantage that assembly work is easier than in the comparative example adopting a configuration in which the insulating ring is formed in a C-ring shape instead of a ring shape closed in the circumferential direction, and the insulating ring is laterally inserted from the side (X direction) of the second region 110.

Although the endoscope according to the embodiment has been described above, the present invention may be improved or modified in some ways without departing from the gist of the present invention.

### EXPLANATION OF REFERENCES

1: endoscope
2: insertion part
3: operating part
4: universal cord
5: soft portion
6: bendable portion
7: distal end portion
8: angle knob
9: angle knob
10: air/water supply button
11: suction button
12: elevating operation lever
13: treatment tool inlet port
14: air/water supply nozzle
16: observation window
17: signal cable
18: illumination window
19: light guide
20: distal end member
22: elevator housing portion
24: opening
26: distal end opening
28: base portion
28A: distal end wall portion
30: flange portion
30A: end surface
32: first wall portion
34: second wall portion
36: elevator
36A: guide surface
38: treatment tool insertion channel
39: treatment tool outlet port
40: cover
42: lever
42A: rotation shaft
44: lever housing chamber
46: operating wire
50: guide wire fixing portion
50A: distal end portion
60: stepped portion
70: insulating ring
70B: insulating ring outer peripheral surface
70C: insulating ring inner peripheral surface
72: flange portion
72A: end surface
72B: end surface
74: cap mounting portion
74A: distal end
74B: outer peripheral surface
76: bendable portion mounting portion
76A: outer peripheral surface
78: flat portion
80: arc portion
100: first region
110: second region
112: cylindrical portion
112A: outer peripheral portion
130: angle rubber
130A: distal end
132: thread
134: adhesive
136: thread winding portion
138: adhesive layer
200: distal end cap
200A: base end
202: upper opening
204: distal end portion opening
206: base end portion opening
208: engaging groove
210: flat portion
212: hook portion

What is claimed is:

1. An endoscope comprising:
a distal end member that is connected to a distal end side of an insertion part and that is made of a conductive material; and
an annular member that is mounted on a base end side of the distal end member and that has an insulating property,
wherein the distal end member has a first region in which an observation window, an illumination window, and a treatment tool outlet port are disposed, and a second region that is provided on a base end side of the first region and on which the annular member is mounted, and
in a case in which the first region and the second region are projected onto a projection plane perpendicular to a longitudinal axis of the insertion part, a maximum outer diameter of the second region is smaller than a maximum outer diameter of the first region.

2. The endoscope according to claim 1,
wherein, in a case in which one of two directions orthogonal to each other on the projection plane is defined as a first direction, and the other of the two directions is defined as a second direction, an outer diameter of the second region in the first direction is smaller than an outer diameter of the first region, and an outer diameter of the second region in the second direction is smaller than an outer diameter of the first region.

3. The endoscope according to claim 2,
wherein the second region is included in the first region on the projection plane.

4. The endoscope according to claim 1,
wherein, in a case in which the annular member is projected onto the projection plane, a minimum inner diameter of the annular member is smaller than the maximum outer diameter of the first region.

5. The endoscope according to claim 1,
wherein the annular member has deformable rigidity, and
the rigidity is rigidity satisfying a condition in which a minimum inner diameter of the annular member is smaller than the maximum outer diameter of the first region even in a case in which the annular member is deformed.

6. The endoscope according to claim 1,
wherein the annular member has a shape closed in a circumferential direction as viewed in a direction of the longitudinal axis.

7. The endoscope according to claim 1,
wherein the annular member has an annular member inner peripheral surface having a shape corresponding to an outer peripheral surface of the second region, and an annular member outer peripheral surface opposite to the annular member inner peripheral surface, and
the annular member outer peripheral surface has an annular protruding portion formed along a circumferential direction, a first outer peripheral surface provided on a base end side with respect to the annular protruding portion, and a second outer peripheral surface provided on a distal end side with respect to the annular protruding portion.

8. The endoscope according to claim 7,
wherein the first outer peripheral surface forms an outer skin mounting surface on which an outer skin of a bendable portion disposed on the base end side of the distal end member is mounted, and
the annular protruding portion has an outer skin abutment surface on which a distal end of the outer skin mounted on the outer skin mounting surface abuts.

9. The endoscope according to claim 8, further comprising:
an outer skin fixing portion that fixes the outer skin mounted on the outer skin mounting surface.

10. The endoscope according to claim 9,
wherein the outer skin fixing portion has a thread winding portion wound around an outer peripheral portion of the outer skin, and an adhesive layer that covers the thread winding portion.

11. The endoscope according to claim 9,
wherein an outer diameter of the annular protruding portion is the same as an outer diameter of the outer skin fixing portion.

12. The endoscope according to claim 7, further comprising:
a distal end cap that is attachably and detachably mounted on the distal end member,
wherein the second outer peripheral surface has a cap mounting surface on which a base end portion of the distal end cap is mounted, and
the annular protruding portion has a cap abutment surface on which a base end of the distal end cap mounted on the cap mounting surface abuts.

13. The endoscope according to claim 12,
wherein an outer diameter of the annular protruding portion is the same as an outer diameter of the base end portion of the distal end cap.

14. The endoscope according to claim 1,
wherein the distal end member has an annular member abutment surface on which a distal end of the annular member mounted in the second region abuts.

15. The endoscope according to claim 14,
wherein the distal end member has a flange portion that protrudes in a radial direction between the first region and the second region, and
the annular member abutment surface is formed by an end surface on a base end side of the flange portion.

16. The endoscope according to claim 15, further comprising:
a distal end cap that is attachably and detachably mounted on the distal end member,
wherein the distal end cap has a first locking portion that is locked to the flange portion.

17. The endoscope according to claim 14,
wherein the distal end member has a stepped portion between the first region and the second region, and
the annular member abutment surface is formed by an end surface on a base end side of the stepped portion.

18. The endoscope according to claim 17, further comprising:
a distal end cap that is attachably and detachably mounted on the distal end member,
wherein the distal end cap has a second locking portion that is locked to the stepped portion.

19. The endoscope according to claim 1,
wherein the distal end member includes
a signal cable that is disposed behind the observation window,
a light guide member that is disposed behind the illumination window, and
a treatment tool insertion channel through which a treatment tool is inserted.

20. The endoscope according to claim 1,
wherein the first region includes an elevator.
